(19) United States
(12) Patent Application Publication
(10) Pub. No.: US 2008/0086195 A1
(43) Pub. Date: Apr. 10, 2008

(54) Polymer-Free Coatings For Medical Devices Formed By Plasma Electrolytic Deposition

(75) Inventors: Liliana Atanasoka, Edina, MN (US); Jan Weber, Maastricht (NL); Robert Warner, Woodbury, MN (US); Steve R. Larsen, Lino Lakes, MN (US)

Correspondence Address: KENYON & KENYON LLP, 1500 K STREET N.W., SUITE 700, WASHINGTON, DC, 20005, US

(73) Assignee: BOSTON SCIENTIFIC SCIMED, INC., Maple Grove, MN (US)

(21) Appl. No.: 11/856,990

(22) Filed: Sep. 18, 2007

Related U.S. Application Data
(60) Provisional application No. 60/849,466, filed on Oct. 5, 2006.

(51) Int. Cl. A61F 2/02; C23C 28/00; C25D 3/66; C25D 5/00; C25D 7/00

(52) U.S. Cl. 623/1.15; 205/188; 205/230; 205/316; 205/80; 424/426; 623/1.42; 623/1.43

(57) ABSTRACT

Methods for the application of a polymer-free coating onto a medical device using plasma electrolytic deposition, comprising: (i) optionally applying a metal precoating onto a medical device; (ii) placing the medical device in an electrolyte solution comprising an electrolyte; and (iii) establishing an electric potential under plasma electrolytic deposition conditions between an electrode and the medical device, such that the plasma electrolytic deposition conditions are adequate to sustain deposition from the electrolyte solution onto the surface of the medical device to form the coating. The invention also relates to coating compositions and coated medical devices, such as stents, made according to these methods. If desired, the polymer-free coating can be a drug-eluting coating.

### RELATED APPLICATIONS

This application claims benefit of U.S. Provisional Application No. 60/849,466, filed Oct. 5, 2006, which is incorporated herein in its entirety.

### FIELD OF THE INVENTION

The field of the present invention is coatings for medical devices, such as stents.

### BACKGROUND

Medical devices such as catheters, guide wires and stents are often made with materials that can cause undesirable complications such as bacterial infection, blood clots, and tissue trauma caused by device insertion. A coating on the medical device can alleviate these challenges without altering the device's bulk material properties. Certain coatings confer a variety of desired properties, such as lubricity, biocompatibility, and antimicrobial action to medical device surfaces. Other coatings can be used to release drugs or make implanted devices more visible to imaging systems. While there are a number of commercially available coating technologies, most use polymers, organic solvents and/or UV curing in the process.

In particular, medical devices such as stents are implantable devices used to maintain the diameter of a vessel after the vessel has been opened or a blockage removed. For example, a stent may be placed in a coronary artery after an angioplasty procedure is performed. Stenting is a growing field of treatment and research in medicine, and various types of stents have found use in a wide range of treatments.

In many applications, it is desirable for implanted stents to become covered in endothelial cells as early as possible after implantation of the stent. This may be particularly true with respect to arterial stenting, and especially coronary arterial stenting. Implanted stents that have not re-endothelialized (i.e., become covered to some degree with endothelial cells) are associated with adverse clinical events such as stent thrombosis. After a stent is implanted it may take several weeks for endothelial cells to propagate from healthy areas within the vessel to the region of the implanted stent and cover the stent.

Stents may be covered with various therapeutic agents to aid acceptance of the stent or to serve other therapeutic goals. For example, stents may be covered with drugs that act to inhibit restenosis (re-blocking) of a vessel. The use of drug-eluting stents has greatly reduced the chance of restenosis.

Many times stents are coated with polymer materials. However, these polymer materials have been found in some patients to be associated with unwanted side effects, such as prolonged inflammatory reactions, for example. For example, the incidence of late occurring thrombotic vessel occlusions and the development of late restenosis have been related to an inflammatory response against non-degradable polymer-coated stent surfaces and/or an incomplete endothelialization. See, e.g., Hausleiter, J. et al., European Heart Journal, 26:1475-1481 (2005). Thus, it would be useful to have methods for making drug-eluting stents having polymer-free stent coatings.

### SUMMARY OF THE INVENTION

The present invention is directed to various methods for making polymer-free coatings for stents and other medical devices using a plasma electrolytic deposition (PED) process. The plasma electrolytic deposition can include plasma electrolytic oxidation (PEO) processes (also known as micro-arc oxidation (MAO), plasma-arc oxidation (PAO) or anodic spark oxidation), as well as plasma electrolytic saturation (PES) processes. In particular, according to certain embodiments of the invention, a process is provided for applying a coating onto a medical device using plasma electrolytic deposition, where the coating provides controlled drug release.

Certain embodiments of the invention relate to methods for the application of a polymer-free drug-eluting coating onto a medical device using plasma electrolytic deposition, comprising: (i) applying an optional metal precoating onto the medical device (e.g., the metal precoating can comprise any suitable metal, such as biodegradable iron or magnesium, as well as non-degradable titanium, or oxides or combinations thereof); (ii) placing the medical device into an electrolyte solution comprising at least one electrolyte (e.g., an ionic form of a drug may be used in certain embodiments); and (iii) establishing an electric potential under plasma electrolytic deposition conditions between a first electrode and the medical device to form a coating. The first electrode may be either a cathode or an anode, depending on the process conditions. The at least one electrolyte can be any chemical compound that ionizes when dissolved to produce an electrically conductive medium. Appropriate plasma electrolytic deposition conditions are used in order to sustain deposition of the coating from the electrolyte solution onto the surface of the medical device to form a polymer-free coating. Furthermore, the plasma electrolytic deposition conditions may be easily adjusted to permit control over the physical properties of the coating, e.g., thickness, porosity, etc.

The medical device to be coated may be made from any conventional material. For example, in the case of stents, common materials could be selected from the group consisting of: iron, magnesium, magnesium composite, magnesium oxide, MP35N, niobium, zirconium, nitinol, tantalum, titanium, tungsten, stainless steel, iridium, platinum, suitable polymers, and mixtures thereof.

Prior to subjecting the medical device to plasma electrolytic deposition conditions, the medical device can be first precoated with a suitable metal in certain embodiments. This precoating preferably comprises a soft metal, for example, selected from magnesium, titanium, aluminum, biodegradable iron, as well as oxides or combinations thereof. If a soft metal or a valve metal is not used, an appropriate metal can be selected, which will provide good coating fracture integrity. The metal precoating may be applied by a hybrid, duplex, or multiplex coating process. The precoating may be applied by a conventional technique such as, but not limited to, a method selected from the group consisting of plating, sputtering, anodization electrodeposition, solvothermal treatment, pulsed laser deposition (PLD) and variations or combinations thereof. The precoating may also be applied to selected portions, for example by means of PLD or by sputtering using a mask, such that that different parts of the stent can be coated with different metal compositions.

In certain embodiments, the coating formed on the medical device may be macroporous, microporous or nanoporous, as well as biodegradable. Advantageously, in certain preferred embodiments, a drug or other bioactive compound may be incorporated into the polymer-free coating. In such cases, the drug or bioactive compound will be released from the coating over time. A few examples of ionic drugs that may be incorporated into the coating using plasma electrolytic deposition include dexamethasone sodium phosphate, paclitaxel and/or methylpyridinium mesylate.

The plasma electrolytic deposition process may be used to easily incorporate additional agents into the coating, either with or without a drug or therapeutic agent. For example, the electrolyte solution can also comprise additional ionic compounds selected from the group consisting of corrosion resistance compounds or growth modifiers, for example. Examples of additional ions may be selected from the group consisting of polyoxometalate, ruthenate, ferrate, chromate, molibdate, silicate, iridate, palatinate, cations for nitriding, cations for carbo-nitriding, and combinations thereof.

The plasma electrolytic deposition conditions can be conveniently adjusted in order to alter the surface morphology and other properties of the coating. Selection of reaction condition parameters can be easily tailored to permit the facile adjustment of coating properties. The plasma electrolytic deposition conditions may be carried out using a suitable regime such as pulsed DC or pulsed AC. For example, voltages of about −100 to 600 V and current densities of 0.5-30 A/dm² may be used. In certain embodiments, the plasma electrolytic deposition conditions could be carried out at a cell voltage of 240-600V, a current density of 0.5-5 A/dm², and a processing time of 5-60 minutes. If instead of DC current, an unbalanced AC is used, there will usually be higher local discharge intensities, which facilitates obtaining high temperature crystal phases, such as anatase or rutile. In that case, preferably a positive voltage regime of up to 500 V could be used, and a negative regime down to −100 V, whereas the current densities may go up to about 30 A/dm², an AC PEO in the range of 10-100 Hz may be used, and processing times of up to 2 minutes. See, e.g., Hanhua Wu, et al., “The Effects of Cathodic and Anodic Voltages on the Characteristics of Porous Nanocrystalline Titania Coatings Fabricated by Microarc Oxidation,” Materials Letters, 59:370-375 (2005)).

Also according to the invention, additional coatings may be optionally applied using techniques such as, but not limited to: nitriding, sputter deposition, electrophoresis, anodization, electrodeposition, solvothermal treatment, and/or hydrothermal treatment, to form one or more multiple films over the medical device.

In various other embodiments, the invention also relates to coatings as well as medical devices and stents that are coated using this process.

### DETAILED DESCRIPTION

The invention relates generally to the application of plasma electrolytic deposition to fabricate a polymer-free coating for a medical device such as a stent. In certain embodiments, the coating produced may be an inorganic, microporous or nanoporous coating that comprises a biologically active agent or drug capable of controlled drug delivery.

Plasma electrolytic deposition methods typically involve the application of different electrical potentials between the medical device and a counter-electrode, which produces an electrical discharge (e.g., a spark or arc plasma micro-discharge) at or near the medical device surface. See A. L. Yerokhin et al., “Plasma Electrolysis for Surface Engineering,” Surface and Coatings Technology, 122:73-93 (1999). Plasma electrolytic deposition (PED) includes plasma electrolytic oxidation processes such as micro-arc oxidation (MAO) also known as plasma-arc oxidation (PAO) or plasma electrolytic oxidation (PEO), as well as the plasma electrolytic saturation (PES) process including plasma electrolytic nitriding (PEN), plasma electrolytic carburizing (PEC) or plasma electrolytic boriding (PEB). In a plasma electrolytic deposition process, spark or arc plasma micro discharges in an aqueous solution are used to ionize gaseous media from the solution such that complex compounds are synthesized on the metal surfaces through the plasma-chemical interactions. Both anode and cathode processes may be used in the present invention.

PES is a technology involved with heating surface discharges in liquid electrolytic plasma. The diffusion of electrolyte into the surface of the electrode can be achieved to saturate the surface with various alloying elements. Both diffusion of elements to the substrate in a saturation process, as well as diffusion outward to the surface in a depletion process have been reported, which are facilitated by the heated surface as well as the plasma envelope around the substrate. The saturation of the surface of the medical device is usually accomplished using electrolyte solutions of simple inorganic acids, suitable salts of the desired ionic species, and certain organic compounds. In certain embodiments, the ionic species for the coating or saturation of the surface will be negatively charged so that they can be drawn into the vapor envelope. It has been reported that metals can be used in such diffusion processes as well, and are often used in the form of solutions with heteropoly acids. See, for example, A. L. Yerokhin et al., “Plasma Electrolysis for Surface Engineering,” Surface and Coatings Technology, 122:73-93 (1999).

In certain embodiments, plasma electrolytic oxidation or micro-arc oxidation is used. Micro-arc oxidation is a variation of traditional electrochemical methods, and has been used for the incorporation of a compact ceramic coating onto a metal (e.g., Al, Ti, Mg, Hf. etc.) or alloy surface. The MAO process combines electrochemical oxidation with a high voltage spark treatment, resulting in a coating formed on the surface. See, e.g., Song et al, Biomaterials, 25:3341(2005); lshizawa and Ogino, J. Biomed Material Research, 29:1071(1995); Li et al., Biomaterials, 25:2867(2004); Zhang et al, J. Biomedical Material Research, 68A:383 (2004); Wang et al, Materials Chemistry and Physics, 90:128 (2005), Guo and An, Applied Surface Science, 246:229 (2005); Wu, C.-T., Surface and Coatings Technology, 166:31-36 (2002) and Meletis et al, Surface and Coating Technology, 150:246-256 (2002).

Generally, the plasma electrolytic deposition techniques combine traditional electrochemical oxidation with a high voltage spark treatment. The plasma electrolytic deposition process had not been generally applicable for conventional medical device materials, such as iron, nitinol, MP35N or stainless steel. However, according to various embodiments of the present invention, a polymer-free coating may be applied using plasma electrolytic deposition techniques to a wide variety of medical devices, including those made from conventional materials.

The surface of the medical device to be treated may be cleaned and/or degreased prior to applying the coating. For example, the surface of the medical device can be polished with an abrasive paper (such as alumina waterproof abrasive paper, for example), then wiping with a suitable solvent, e.g., acetone, ethyl alcohol and/or distilled water. Alternatively, in certain embodiments, the medical device will simply be rinsed with distilled water and allowed to air dry.

Prior to the plasma electrolytic deposition treatment, the surface of the medical device may optionally be covered with a metal (metal oxide or any ceramics) pre-coating layer if desired. The metal precoating may be applied by conventional methods such as plating, sputtering, vapor deposition (i.e., chemical, physical, plasma enhanced physical, or thermal spraying, etc.), or combinations thereof. The material for the precoating should be one that is suitable for subsequent plasma electrolytic deposition. For example, the so-called soft or valve metals may be used in certain preferred embodiments. Typically, metals such as aluminum, titanium, magnesium, zirconium and hafnium can be used as a precoating on the medical device prior to the plasma electrolytic deposition treatment. The precoating metal may comprise, for example, oxides and/or composites thereof, e.g., Al₂O₃—SiO₂, Al₂O₃—MgO, Al₂O₃—CaO, and others.

After the precoating has been applied, the polymer-free coating may be applied to the medical device under plasma electrolytic deposition conditions. If desired, the medical device could be patterned or treated in order to provide a masked or template-based synthesis of the plasma electrolytic deposition coating. In this embodiment, certain areas of the medical device are masked in order to apply different types of coatings or different drugs to specific regions on the surface of the medical device. See, e.g., Lee, W. et al., Angew. Chem. Int. Ed., 44:6050-6054 (2005); Datta, M. et al., Electrochimica Acta, 45:2535-2558 (2000) and Volkel, B. et al., Surface Science, 597:32-41 (2005). This approach may be useful in order to combine different therapeutic benefits from two or more separate components, such as different drugs combinations. In certain embodiments, for example, it would be possible to have one part of the medical device immobilized with heparin for anticoagulation, while another surface will be immobilized with a second drug, for example, FK506 (tacrolims) for the prevention of neointimal hyperplasia. However, other combinations of drugs and coatings are also possible according the processes of the invention, and can be customized as desired for optimal results and therapeutic benefit.

The plasma electrolytic deposition process is carried out in an electrolyte solution connected to a power supply. The setup will be very similar in configuration to a conventional anodic oxidation or electroplating process, but one notable difference is that the applied electrode potential in the plasma electrolytic deposition process will be much higher. See, e.g., Meletis, E. I., et al., Surface and Coatings Technology, 150:246-256 (2002). The plasma electrolytic deposition process involves electrolysis by applying an electrical potential between the medical device to be coated and the counter-electrode, as well as the production of an electrical discharge in close proximity to the medical device surface. One of the benefits of plasma electrolytic deposition is that environmentally friendly solutions may be used. For example, the electrolyte preferably uses distilled water as the solvent.

Plasma electrolytic deposition is normally carried out in an electrolyser with a high power electric source. For example, the electrolyser can be a water-cooled bath placed on a dielectric base and confined in a grounded steel frame, which an insulated current supply. To deposit the coatings, the medical device to be coated is attached to the current supply and typically either immersed in the electrolyte or dripped with electrolyte, e.g., as shown in FIG. 1b of Meletis, E. I., et al., Surface and Coatings Technology, 150:246-256 (2002).

For example, in a certain embodiment, the medical device can be connected to the positive terminal (anode) and a nonreactive metal, such as stainless steel, is connected to the negative terminal (cathode). Both the anode and the cathode are immersed into the electrolyte solution, and the voltage applied across them. A suitable voltage can be applied and the power supply can be adjusted as necessary for the optimal current and amplitudes of the anode and cathode voltages.

In other embodiments, either DC or AC sources may be applied, including DC sources, pulsed DC sources, unbalanced AC sources (i.e., alternating current with different amplitudes to the positive and negative components), heteropolar pulsed current, and combinations thereof. Each of these electric sources may be optimized to achieve the desired coating and/or surface characteristics. The parameters will vary depending on the composition of the electrolyte solution and medical device, etc., but can be estimated using standard calculations as set forth, for example, in A. L. Yerokhin et al., “Kinetic Aspects of Aluminum Titanate Layer Formation on Titanium Alloys by Plasma Electrolytic Oxidation,” Applied Surface Science, 200:172-184(2002). Current density is often set within the range of 0.01 to 0.3 A/cm², which usually provides an acceptable coating growth rate.

The final coating on the medical device should be optimized in terms of chemical composition, surface roughness, surface energy and porosity, etc. to provide good cell adhesion and cell proliferation. For example, it is possible to have nanosized porosity, which can be especially useful for applications where slow release of embedded drugs might be desired. The drug release profile will be controlled by a number of factors in addition to porosity, including wetting and surface energy. Increased wetting and surface energy improves a material's adhesion characteristics, thereby allowing improved release characteristics. The drug release will need to be optimized to give a desired profile for a particular bioactive agent and coating system. See, e.g., Zhang, Y. M. et al., Osteoblast Behavior on MAO Titanium, 383-391 (2003). Fortunately, the properties of the coating made by the plasma electrolytic deposition technique(s) may be tailored by easily adjusting the process conditions such as, but not limited to: the applied current densities, concentration and constituents of the electrolyte, processing time, current and voltage. See, e.g., Guo, H. F., et al., Applied Surface Science, 246:229-238 (2005); Ishizawa, H., Journal of Biomedical Materials Research, 35:199-206 (1997); Li, L.-H. et al., Biomaterials, 25:2867-2875 (2004); Nakayama, Y., et al., Cardiovascular Radiation Medicine, 4:77-82 (2003); Nie, X., et al., Surface and Coatings Technology, 125:407-414 (2000); Oh, H.-J., et al., Surface & Coatings Technology, 198:247-252 (2005); Ryu, H. S., et al., Current Applied Physics, 5:512-515 (2005); Song et al., Biomaterials, 25:3341-3349 (2005); Sundararajan, G., Surface and Coatings Technology, 167:269-277 (2003); Wang, Y. M. et al., Materials Chemistry and Physics, 90:128-133 (2005); and Wu, H., et al., Materials Letters, 59:370-375 (2005).

The electrolyte solution will contain the desired ions in solution. A variety of ions may be incorporated into the coating, as desired to confer beneficial properties. In certain embodiments, an ionic drug is present to be incorporated into the polymer-free coating. In certain other embodiments, additional components may confer desirable properties such as corrosion resistance and control of drug release, better tribological properties (resistant to friction and wear), increased growth rates, or other functional requirements. For example, ions such as polyoxometalate, ruthenate, ferrate, chromate, molibdate, or silicate may be used. Such ions may be incorporated in the electrolyte solution with the ionic drug. For example, ions such as polyoxometalate, ruthenate, ferrate, chromate, molibdate, silicate, iridate, palatinate, cations for nitriding, cations for carbo-nitriding, etc. may be used to impart corrosion resistance and control.

The electrolyte solution is preferably maintained at a temperature less than the boiling point of the solvent used. For example, a temperature range of about 40° C.-80° C. for an aqueous system may be conveniently used. In other embodiments, the temperature can be maintained greater than about 80° C. Depending upon the solvent, and when incorporating a drug in situ, often the plasma electrolytic deposition conditions are then carried out at a temperature of less than about 200° C., otherwise it is possible to raise the temperature greater than about 200° C.

The temperature may be automatically controlled by an external source. For example, a heat exchanger or refrigeration equipment may be used in order to regulate the temperature in certain embodiments.

The electrolyte solution is conveniently kept within a pH of about 6-12, preferably about 12-13. However, any suitable pH may be used. See, e.g., Yong Han, et al., “Structure and in vitro Bioactivity of Titania-Based Films by Micro-Arc Oxidation,” Surface and Coatings Technology, 168:249-258 (2003).

By applying a high current/high voltage the plasma discharges at the metal/electrolyte interface. As the plasma electrolytic deposition process takes place, an oxide film is formed initially by electrochemical reaction, but when using a high current and high voltage, this superficial dielectric layer is broken down. Typical voltages are in the range of 200V-600V. The selection of voltage will have an effect on the phase and surface morphology of the coating. See, for example, Song et al., Biomaterials, 25:3341(2005), FIG. 4.

It is common to see intensive gas evolution and sparking phenomenon at the surface. The voltage and time corresponding to the appearance of sparks on the surface is referred to as the breakdown voltage (typically about 240V to about 440V) and the ignition time (typically about 40-300 seconds).

The resulting plasmas appear as sparks moving across the surface, which induce the evolution of the plasmas, where over time the sparks become microarcs, and then arcs. The plasmas in turn oxidize the surface. Also, local conditions of heat and pressure sinter and anneal the coating. The micro-arc plasmas may be conveniently monitored using optical emission spectroscopy.

The electric field applied must be greater than the dielectric breakdown field for the oxide. It is important to maintain a sufficient current in order to have good control of the process. Over time, the resistance of the sample surface increases over time due to the growing coating layer, and the current may drop. The coating is typically from about 1 to about 50 microns thick. For example, the thickness is preferably from about 1 to about 10 microns, and more preferably from about 2 to about 5 microns. The process may be monitored using optical emission spectroscopy to determine the dominant species present in the arcs. After the reaction is completed, the medical device may be washed with distilled water and dried.

Since the plasma electrolytic deposition process is performed in an electrolyte solution, it offers the opportunity of easily incorporating various functional ions into the surface layer, by controlling the composition and concentration of the electrolyte. The coating properties such as thickness, porosity, roughness, etc. can be precisely controlled by the plasma electrolytic deposition process parameters of voltage, current, DC, AC, pulse parameters, number of steps, time, electrolyte concentration, pH, and temperature. For example, rapid cooling will result in a complex mixture of amorphous material and nanocrystalline phases. On the other hand, prolonged reaction times may lead to a decrease in porosity.

It is also possible to perform the plasma processing before cutting the medical device. For instance, the plasma processing could be performed on a tube from which the medical device is cut to provide a porous layer on the outside of this tube as described herein. After providing such a porous coating, the stent pattern can be cut using an ablating laser, such as a femto second laser. Preferably, the process does not give additional debris and hardly any heat generation, so that any drug included in the porous coating is not affected. In this embodiment, preferably an abluminal coating is achieved, where the inner surface is less rough than with an all-around coating, which could cause pinholes in the balloon delivery system.

Thus, according to the invention, plasma electrolytic deposition can be used to form polymer-free coatings on various medical devices, including stents. In certain preferred embodiments, drug-eluting coatings are also provided. The ceramic coating obtained by plasma electrolytic deposition has extremely high adhesion, good hardness properties, high erosion and abrasion wear resistance, and good dielectric properties. Also, the plasma electrolytic deposition produces a thick, well bonded ceramic coating on a variety of reactive light metal alloys and could be used in place of more expensive materials or heavier materials.

It is also within the scope of the present invention to apply multiple layers of coatings onto the medical device. Such multiple layers may contain the same or different therapeutic agents and/or additional materials. These additional coatings may be applied by methods known in the art, such as nitriding, sputter deposition, electrophoresis, hydrothermal treatment, etc.

The surface morphology and phase structure of the final coating may be analyzed using any appropriate technique, such as optical emission spectroscopy (OES), scanning electron microscope (SEM) or X-ray powder diffraction (XRD).

The medical device may also contain a radio-opacifying agent within its structure to facilitate viewing the medical device during insertion and at any point while the device is implanted. Non-limiting examples of radio-opacifying agents are bismuth subcarbonate, bismuth oxychloride, bismuth trioxide, barium sulfate, tungsten, and mixtures thereof.

The therapeutic agent may be any ionic pharmaceutically acceptable agent such as a non-genetic therapeutic agent, a biomolecule, a small molecule, or cells. Combinations of different drugs may be used on the medical device. Exemplary therapeutic agents include anti-thrombogenic agents such heparin, heparin derivatives, prostaglandin (including micellar prostaglandin E1), urokinase, and PPACK (dextrophenylalanine proline arginine chloromethylketone); anti-proliferative agents such as enoxaprin, angiopeptin, sirolimus (rapamycin), tacrolimus, everolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis(2-aminoethyl)ethyleneglycol-N,N,N′,N′-tetraacetic acid and mixtures thereof, antibiotics such as gentamycin, rifampin, minocyclin, and ciprofolxacin; antibodies including chimeric antibodies and antibody fragments; anesthetic agents such as lidocaine, bupivacaine, and ropivacaine; nitric oxide; nitric oxide (NO) donors such as linsidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promoters such as growth factors, transcriptional activators, and translational promoters; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; inhibitors of heat shock proteins such as geldanamycin; angiotensin converting enzyme (ACE) inhibitors; beta-blockers; bAR kinase (bARKct) inhibitors; phospholamban inhibitors; protein-bound particle drugs such as ABRAXANE™; and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes. The therapeutic agent could also be a polymer-drug conjugate, such as paclitaxel-polyglutamate or everolimus polyglutamate, for example.

Non-limiting examples of proteins include serca-2 protein, monocyte chemoattractant proteins (“MCP-1) and bone morphogenic proteins (“BMP's”), such as, for example, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15. Preferred BMPS are any of BMP-2, BMP-3, BMP-4, BMP-5, BMP-6, and BMP-7. These BMPs can be provided as homdimers, heterodimers, or combinations thereof, alone or together with other molecules. Alternatively, or in addition, molecules capable of inducing an upstream or downstream effect of a BMP can be provided. Such molecules include any of the “hedghog” proteins, or the DNA's encoding them. Non-limiting examples of genes include survival genes that protect against cell death, such as anti-apoptotic Bcl-2 family factors and Akt kinase; serca 2 gene; and combinations thereof. Non-limiting examples of angiogenic factors include acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor, and insulin like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase (“TK”) and combinations thereof and other agents useful for interfering with cell proliferation.

Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered. Non-limiting examples of cells include side population (SP) cells, lineage negative (Lin⁻) cells including Lin⁻ CD34⁻, Lin⁻CD34⁺, Lin⁻cKit⁺, mesenchymal stem cells including mesenchymal stem cells with 5-aza, cord blood cells, cardiac or other tissue derived stem cells, whole bone marrow, bone marrow mononuclear cells, endothelial progenitor cells, skeletal myoblasts or satellite cells, muscle derived cells, go cells, endothelial cells, adult cardiomyocytes, fibroblasts, smooth muscle cells, adult cardiac fibroblasts+5-aza, genetically modified cells, tissue engineered grafts, MyoD scar fibroblasts, pacing cells, embryonic stem cell clones, embryonic stem cells, fetal or neonatal cells, immunologically masked cells, and teratoma derived cells.

Any suitable polymer-drug conjugate may also be used. The macromolecules used for the preparation of the conjugate should be selected to be pharmaceutically acceptable, e.g., water-soluble, nontoxic, and nonimmunogenic molecules, with suitable functional groups for attaching the therapeutic agent or drug. Examples of suitable polymers include HPMA, PEG, poly(glutamic acid) (PG), and albumin. The term “polymer-drug conjugate” includes a biologically acceptable polymer in combination with a therapeutic agent, and includes polymer-protein conjugates as well as polymeric micelles comprising a therapeutic agent. Examples of polymer-drug conjugates that may be used include paclitaxel-polyglutamate conjugates, everolimus-polyglutamate conjugates, doxorubicin-HPMA copolymer conjugates, and polyethylene glycol (PEG)-camptothecin conjugates. See, e.g., Ruth Duncan, “The Dawning Era of Polymer Therapeutics,” Nature Reviews: Drug Discoverv, 2:347-360 (May 2003) and Rainer Haag and Felix Kratz, “Polymer Therapeutics: Concepts and Applications,” Angew. Chem. Int. Ed., 45:1198-1215 (2006).

Any of the therapeutic agents may be combined to the extent such combination is biologically compatible.

All of the above-mentioned publications, patents and patent applications are herein incorporated by reference in their entirety to the same extent as if each individual publication, patent or patent application was specifically and individually indicated to be incorporated by reference in its entirety.

While the present invention has been described with reference to what are presently considered to be preferred embodiments thereof, it is to be understood that the present invention is not limited to the disclosed embodiments or constructions. On the contrary, the present invention is intended to cover various modifications and equivalent arrangements.

What is claimed is:

1. A method for the application of a polymer-free coating onto a medical device using a plasma electrolytic deposition process, comprising:
(i) optionally applying a metal precoating onto the medical device;
(ii) placing the medical device in an electrolyte solution containing at least one electrolyte; and
(iii) establishing an electric potential under plasma electrolytic deposition conditions between a first electrode and the medical device,
wherein the plasma electrolytic deposition conditions are sufficient to sustain deposition of at least one electrolyte from the electrolyte solution onto the surface of the medical device to form a polymer-free coating.

2. The method of claim 1, wherein the plasma electrolytic deposition process is selected from the group consisting of a micro-arc oxidation (MAO) process, a plasma-arc oxidation (PAO) process, a plasma electrolytic saturation process, and combinations thereof.

3. The method of claim 1, wherein the plasma electrolytic deposition process is a plasma electrolytic saturation process.

4. The method of claim 3, wherein the plasma electrolytic saturation process is selected from the group consisting of plasma electrolytic nitriding, plasma electrolytic carburizing, plasma electrolytic boriding, and combinations thereof.

5. The method of claim 1, wherein the medical device comprises a material selected from the group consisting of: iron, magnesium, magnesium composite, magnesium oxide, MP35N, niobium, zirconium, nitinol, tanatalum, titanium, tungsten, stainless steel, iridium, platinum, and mixtures thereof.

6. The method of claim 1, wherein the metal precoating is applied by a hybrid, duplex, or multiplex coating process.

7. The method of claim 1, wherein the metal precoating is applied by a method selected from the group consisting of plating, sputtering, anodization, electrodeposition, solvothermal treatment, and combinations thereof.

8. The method of claim 1, wherein the precoating comprises biodegradable iron, magnesium, magnesium oxide, or combinations thereof.

9. The method of claim 1, wherein the precoating comprises biodegradable iron.

10. The method of claim 1, wherein the polymer-free coating formed on the medical device is macroporous.

11. The method of claim 1, wherein the polymer-free coating formed on the medical device is microporous.

12. The method of claim 1, wherein the polymer-free coating formed on the medical device is nanoporous.

13. The method of claim 1, wherein the polymer-free coating formed on the medical device is biodegradable.

14. The method of claim 1, wherein the at least one electrolyte is a therapeutic agent.

15. The method of claim 14, wherein the therapeutic agent is selected from the group consisting of an anti-thrombogenic agent, an anti-proliferative agent, an anti-inflammatory agent, an anti-neoplastic agent, an anti-mitotic agent, an anti-cancer agent, an anti-microbial agent, a prostaglandin, a biofilm synthesis inhibitor, an antibody, a non-steroidal anti-inflammatory agent, a chelating agent, an antibiotic, an anesthetic agent, a nitric oxide (NO) donor, an anti-coagulant, a platelet aggregation inhibitor, an antithrombin compound, an anti-restenosis agent, a vascular cell growth promoter, a vascular cell growth inhibitor, an inhibitors of heat shock protein, a cephalosporin, an aminoglycoside, an antisense inhibitor of c-myc oncogene, a monoclonal antibody agent capable of blocking smooth muscle cell proliferation, a tick antiplatelet factors, a growth factor, a transcriptional activator, a translational promoter, a growth factor inhibitor, a growth factor receptor antagonist, a transcriptional repressor, a translational repressor, a replication inhibitor, an inhibitory antibody, an antibody directed against growth factors, a bifunctional molecules consisting of a growth factor and a cytotoxin, a bifunctional molecules consisting of an antibody and a cytotoxin, a cholesterol-lowering agent, a vasodilating agent, an agent that interferes with endogenous vascoactive mechanisms, an RGD peptide-containing compound, a platelet receptor antagonist, an anti-thrombin antibody, an anti-platelet receptor antibody, an angiotensin converting enzyme (ACE) inhibitor, a beta-blocker, a bAR kinase (bARKct) inhibitor, a phospholamban inhibitor, a protein-bound particle drug, and combinations thereof.

16. The method of claim 14, wherein the therapeutic agent is selected from the group consisting of heparin, a heparin derivative, a micellar prostaglandin E1, a urokinase, PPACK (dextrophenylalanine proline arginine chloromethylketone), enoxaprin, angiopeptin, sirolimus (rapamycin), tacrolimus, everolimus, hirudin, acetylsalicylic acid, dexamethasone, rosiglitazone, prednisolone, corticosterone, budesonide, estrogen, estrodiol, sulfasalazine, acetylsalicylic acid, mycophenolic acid, mesalamine, paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, angiostatin, triclosan, nitrofurantoin, ethylenediaminetetraacetic acid, O,O′-bis(2-aminoethyl)ethyleneglycol-N,N,N′,N′-tetraacetic acid, gentamycin, rifampin, minocyclin, ciprofolxacin, lidocaine, bupivacaine, and ropivacaine, nitric oxide, linsidomine, molsidomine, L-arginine, NO-carbohydrate adducts, polymeric or oligomeric NO adducts, D-Phe-Pro-Arg chloromethyl ketone, enoxaparin, hirudin, warfarin sodium, Dicumarol, aspirin, prostaglandin inhibitors, cilostazol, geldanamycin, ABRAXANE™, and combinations thereof.

17. The method of claim 14, wherein the therapeutic agent is selected from the group consisting of peptides, polypeptides, proteins, oligonucleotides, nucleic acids, antisense nucleic acids, small interfering RNA (siRNA), ribozymes, genes, carbohydrates, angiogenic factors, cell cycle inhibitors, stem cells, progenitor cells, endothelial cells, adult cardiomyocytes, smooth muscle cells, and combinations thereof.

18. The method of claim 14, wherein the therapeutic agent is a consisting of polymer-drug conjugate.

19. The method of claim 18, wherein the polymer-drug conjugate is selected from the group consisting of paclitaxel-polyglutamate conjugates, everolimus-polyglutamate conjugates, doxorubicin-HPMA copolymer conjugates, polyethylene glycol (PEG)-camptothecin conjugates, and mixtures thereof.

20. The method of claim 1, wherein the electrolyte solution further comprises additional ionic compounds selected from the group consisting of corrosion resistance compounds.

21. The method of claim 1, wherein the electrolyte solution further comprises additional ions selected from the group consisting of polyoxometalate, ruthenate, ferrate, chromate, molibdate, silicate, iridate, palatinate, cations for nitriding, cations for carbo-nitriding, and combinations thereof.

22. The method of claim 1, wherein the plasma electrolytic deposition conditions are carried out using pulsed DC or pulsed AC.

23. The method of claim 1, wherein the plasma electrolytic deposition conditions are carried out at a cell voltage of about −100 V to about 600V.

24. The method of claim 1, wherein the plasma electrolytic deposition is carried out at a current density of about 0.5 to about 30 A/dm².

25. The method of claim 1, wherein the plasma electrolytic deposition is carried out in an alkaline electrolyte.

26. The method of claim 1, wherein the plasma electrolytic deposition is carried out in an aqueous electrolyte and the temperature is maintained at a temperature of less than about 80° C.

27. The method of claim 1, wherein the plasma electrolytic deposition is carried out at a temperature of greater than about 200° C.

28. The method of claim 1, wherein the plasma electrolytic deposition is carried out at a temperature of less than about 200° C.

29. The method of claim 1, wherein the plasma electrolytic deposition is carried out in a non-aqueous electrolyte and the temperature is maintained at less than the boiling point of the non-aqueous electrolyte.

30. The method of claim 1, wherein the plasma electrolytic deposition is carried out for a processing time of about 2 minutes to about 60 minutes.

31. The method of claim 30, wherein the plasma electrolytic deposition is carried out for a processing time of about 2 minutes to about 15 minutes.

32. A process of claim 1, further comprising at least one additional coating that is applied using a technique selected from the group consisting of: nitriding, sputter deposition, electrophoresis, plasma immersion ion implantation, micro-plasma treatment, nanoplasma treatment, and hydrothermal treatment.

33. A coating formed by the process of claim 1.

34. A coating of claim 33, wherein the coating provides controlled release of a drug or bioactive agent.

35. A medical device comprising the coating of claim 33.

36. A catheter, guide wire or stent comprising the coating of claim 33.

37. A method for making a medical device, comprising:
(i) providing a metal substrate;
(i) optionally applying a metal precoating onto the metal substrate;
(ii) placing the metal substrate in an electrolyte solution containing at least one electrolyte;
(iii) establishing an electric potential under plasma electrolytic deposition conditions between a first electrode and the medical device; wherein the plasma electrolytic deposition conditions are sufficient to sustain deposition of at least one electrolyte from the electrolyte solution onto the surface of the medical device to form a polymer-free coating; and
(iv) forming the metal substrate into a medical device.
